(19) United States
(12) Patent Application Publication
(10) Pub. No.: US 2023/0181885 A1
(43) Pub. Date: Jun. 15, 2023

(54) IMPLANT AND HANDHELD MEDICAL DEVICE

(71) Applicant: OLYMPUS WINTER & IBE GMBH, Hamburg (DE)

(72) Inventors: Christian BROCKMANN, Hollenstedt (DE); Thorsten JÜRGENS, Hamburg (DE)

(73) Assignee: OLYMPUS WINTER & IBE GMBH, Hamburg (DE)

(21) Appl. No.: 18/075,019

(22) Filed: Dec. 5, 2022

Related U.S. Application Data
(60) Provisional application No. 63/288,879, filed on Dec. 13, 2021.

(51) Int. Cl. A61M 29/02

(52) U.S. Cl. CPC A61M 29/02; A61M 2205/3317

(57) ABSTRACT

An implant and a handheld medical device using which the implant may be removed particularly easily and without traumatizing the patient. This is achieved in that a proximal end of an implant has a coupling part having a ferromagnetic element, the implant being able to be withdrawn from the urethra via this coupling part.

The invention relates to an implant according to the preamble of claim 1. Furthermore, the invention relates to a handheld medical device according to the preamble of claim 10.

Various methods or technologies are known for the treatment of benign prostatic hyperplasia (BPH). In a minimally invasive treatment of BPH symptoms which is particularly gentle to the body, a removable implant is temporarily placed in the urethra or in the prostatic part of the urethra of the patient. Such an implant is a wire structure made of a shape memory alloy, such as Nitinol. The wire structure is pushed in a collapsed state by a catheter into the correct position to unfold there into its predetermined basic structure. This structure, which can be formed from three or four wires, is a basket structure. This basket structure expands the urethra in its unfolded basic structure. The stricture tissue of the urethra is denatured in the course of a few days by the expansion of the wire structure against the tissue of the urethra. This denaturing of the tissue takes place due to the ischemic pressure of the individual wires on the cells of the tissue, which leads to reduced or entirely absent perfusion. As a result, an oxygen deficiency occurs in the cells due to the lack of perfusion and finally the cells die. Within a few days, the tissue may be reduced in such a way that the urine flow nearly normalizes. After ending this treatment, the implant may be retrieved from the urethra again by means of a catheter.

In the above-described technology, the implant has a thread or a suture at a proximal end. To retrieve the implant from the urethra, the catheter is led over this thread and thus guided to the proximal end of the implant. By pulling on the thread, the implant is folded or drawn into the catheter. At the same time, the wire structure collapses in such a way that it has space in the catheter. It has proven to be disadvantageous that during the entire treatment phase the thread remains in the urethra or protrudes out of the urethra. This is not only unpleasant, but can also result in infections, due to which the treatment has to be terminated.

Proceeding therefrom, the invention is based on the object of providing an implant and a handheld medical device using which the implant may be removed particularly easily and without traumatizing the patient.

An achievement of this object is described by the features of claim 1. It is accordingly provided that a proximal end of an implant has a coupling part having a ferromagnetic element, the implant being able to be withdrawn from the urethra via this coupling part. This coupling part is substantially shorter than the above-described thread and can therefore be located entirely in the urethra. To retrieve the implant from the body, it is contacted within the urethra by a handheld medical device via the coupling part and drawn out. Upon this contacting or coupling between the coupling part of the implant and the handheld medical device, a magnetic force acts between the ferromagnetic element of the implant and the handheld medical device. For this purpose, the handheld medical device also has a corresponding ferromagnetic counter element or a magnet at a distal end. Because of the ferromagnetic element of the implant, an attractive effect forms between the coupling part of the implant and the handheld medical device. It is thus possible that the implant is contacted or the required coupling takes place between the implant and the handheld medical device. The magnetic attraction force is used here, in particular for correct positioning of the coupling part relative to the handheld medical device. It is thus possible due to the ferromagnetic element inside the coupling part to contact the implant and to retrieve it in a particularly simple manner that is gentle to the body.

In particular, it is provided according to the invention that the ferromagnetic element is a permanent magnet. This permanent magnet has the effect that the coupling part of the implant has an attractive effect on ferromagnetic elements in its surroundings. Thus, for example, a magnetic or a magnetizable distal end of a handheld medical device is attracted by the permanent magnet. This permanent magnet can be formed as a bar magnet and have a size of one or a few millimeters. The advantage of a permanent magnet is that the magnetic polarization is fixed. The coupling part of the implant may therefore be contacted with a corresponding ferromagnetic object, so that an attractive effect forms between the elements.

Furthermore, it can be provided according to the invention that the ferromagnetic element is the coupling part itself. For this purpose, the coupling part, which is connected to the implant, is formed as the ferromagnetic element or permanent magnet. In addition, it is conceivable that the element has a coating to be protected from external chemical influences. However, it is similarly also conceivable that the ferromagnetic element is integrated in the coupling part, in particular enclosed by the coupling part.

One exemplary embodiment could provide that a permanent magnet or a ferromagnetic element is extrusion-coated by a coupling part. In this example, the coupling part is produced from a plastic which flows around the ferromagnetic element during the production and is formed in such a way that the magnetic field lines outside the coupling part are still strong enough for the above-mentioned purposes.

It is preferably conceivable that the coupling part has at least one projection, one slot, one notch, one bore, one pin, or the like as mechanical coupling means. In addition to the magnetic coupling, the implant can form a mechanical connection by form fit with a corresponding counterpart of the handheld medical device. For such a mechanical connection, the coupling part can have at least one projection, one slot, one notch, one bore, or one pin. It is thus conceivable that, for example, corresponding teeth or a slot or a web or a bore or a pin mechanically interlock and are additionally strengthened or held in position by magnetic force. A particularly advantageous exemplary embodiment of the invention can provide that the implant is initially positioned relative to the handheld medical device by the magnetic effect, in order to then form the mechanical connection. Due to the combined mechanical and magnetic force, the implant can then be removed in a gentle and simple manner from the urethra.

Furthermore, it can be provided according to the invention that the coupling part of the implant has a circular cross section or a cross section like a segment of a circle. The transport through the urethra becomes particularly simple and gentle to the body due to this shape. Any projections or edges could result in traumatization of the patient and are therefore to be avoided.

One particularly advantageous exemplary embodiment of the invention can provide that a sensor and/or a data memory are arranged in the coupling part, in particular that a sensor and/or a data memory are integrated in the coupling part. The implanted implant may be identified by this sensor or by this data memory. For this purpose, corresponding identifiers or items of information are stored on the data memory, which can give information about the implant and the patient. Items of information about the implant and the patient may thus be verified by contactless readout by means of a corresponding scanner or in contact with the handheld medical device. In addition, it is conceivable that it is detectable via the sensor, which can be a Reed sensor or a metal sensor, such as a Hall probe, whether the coupling of the coupling part to a coupling counterpart is established. Both the sensor and the data memory can be a passive component to thus keep the structural size as small as possible.

It is conceivable according to the invention that the coupling part is connected via a wire or a thread or directly to the wire structure of the implant. In particular the length of the wire or of the thread is to be kept as short as possible here. While a direct connection of the coupling part to the wire structure represents a very compact implant, a higher level of flexibility of the implant may be produced by a wire or a thread, which could ultimately be more pleasant for the patient. The length of the thread or else of the wire between the coupling part and the wire structure can be a few millimeters, in particular 1 mm to 5 mm or 1 mm to 10 mm, or 1 cm to 2 cm or even 1 cm to 3 cm. To retrieve the implant, it can prove to be advantageous if the coupling part is connected via a thread or wire to the wire structure for the coupling or positioning of the coupling part in relation to a coupling counterpart. On the other hand, the positioning or placement of the wire structure is made simpler if the coupling part is connected directly to the wire structure. This is because the wire structure can then be shifted via the coupling part directly into the correct position.

A handheld medical device for achieving the stated object has the features of claim 10. It is accordingly provided that a handheld medical device for positioning and retrieving an implant in a urethra or out of a urethra has a rod, which is in particular flexible. This rod is insertable via a catheter into the urethra. At a distal end, the rod has a coupling counterpart. The implant is designed as claimed in at least one of claims 1 to 9. The essential feature of the handheld medical device according to the invention is that the coupling counterpart has a ferromagnetic counter element for coupling to a coupling part of the implant. The implant may be contacted in a very reliable manner which is gentle to the body and removed from the human body by this handheld medical device. A guided coupling or positioning of the coupling counterpart to the coupling part takes place due to the ferromagnetic embodiment of the counter element. Because of the magnetic interaction between the implant and the handheld device, the treating person only has to move the distal end of the rod into the vicinity of the proximal end of the implant. The coupling counterpart and the coupling part then join together in their coupling positions due to their attractive effect. The positioning of the implant in the urethra is similarly made particularly advantageous. During the pushing-in, the contact between the coupling part and the coupling counterpart or between the rod and the implant is strengthened by the magnetic interaction. There is therefore not only a mechanical connection but also a magnetic connection between the implant and the rod, which enables secure placement of the implant in the urethra. The magnetic field strength, however, permits the two above-mentioned parts to be separated from one another by a slight force action or by a minor rotation of the rod.

One advantageous refinement of the handheld medical device can provide that the ferromagnetic counter element is a permanent magnet. This permanent magnet has the effect that the coupling counterpart of the rod has an attractive effect on the ferromagnetic element in its surroundings. Thus, for example, a magnetic or a magnetizable distal end of the implant is attracted by the permanent magnet. This permanent magnet can be formed as a bar magnet and have a size of one or a few millimeters. The advantage of a permanent magnet is that the magnetic polarization is fixed. The coupling counterpart of the rod may therefore be contacted with a corresponding ferromagnetic object, so that an attractive effect forms between the elements.

It can preferably furthermore be provided that the ferromagnetic counter element is the coupling counterpart. For this purpose, the coupling counterpart, which is connected to the rod, is formed as the ferromagnetic counter element or permanent magnet. In addition, it is conceivable that the counter element has a coating to be protected from external chemical influences. Similarly, however, it is also conceivable that the ferromagnetic counter element is integrated in the coupling counterpart, in particular enclosed by the coupling counterpart. One exemplary embodiment could provide that a permanent magnet or a ferromagnetic counter element is extrusion-coated by a coupling counterpart. In this example, the coupling counterpart is produced from a plastic which closes around the ferromagnetic counter element and is formed in such a way that the magnetic field lines outside the coupling counterpart are still strong enough for the above-mentioned purposes.

One particularly advantageous exemplary embodiment of the invention can provide that the coupling counterpart has at least one projection, one slot, one notch, one bore, one pin, or the like as mechanical coupling means, this coupling means being complementary to the coupling means of the coupling part. In addition to the magnetic coupling, the rod can form a mechanical connection by form fit with a corresponding counterpart of the implant. For such a mechanical connection, the coupling counterpart can have at least one projection, one slot, one notch, one bore, and/or one pin. It is thus conceivable that, for example, corresponding teeth or a slot or a notch or a bore or a pin mechanically interlock and are additionally strengthened or held in position by magnetic force.

One particularly advantageous exemplary embodiment of the invention can provide that the implant is initially positioned relative to the handheld mechanical device by the magnetic effect, in order to then form the mechanical connection. The implant may then be retrieved in a gentle and simple manner from the urethra by the combined mechanical and magnetic force.

Furthermore, it can be provided according to the invention that the coupling part of the implant and the coupling counterpart of the rod have a shared circular or circle-like cross section in the assembled state. The transport through the urethra is made particularly simple and gentle to the body due to this shape. Any projections or edges could result in traumatization of the patient and are therefore to be avoided. Coupling part and coupling counterpart can thus be drawn together into the catheter. In the interior of the catheter, the coupling is thus secured against radial displacement. It is thus possible to pull with greater force without inadvertently detaching the magnetic coupling.

One particularly advantageous exemplary embodiment of the invention can provide that a sensor and/or a data memory are arranged in the coupling counterpart, in particular that a sensor and/or a data memory are integrated in the coupling counterpart. The implanted implant may be identified by this sensor or by this data memory. For this purpose, corresponding identifiers or items of information are stored on the data memory, which can provide information about the implant, the handheld device used, and the patient. Items of information about the handheld device and the patient may thus be verified by a contactless readout by means of a corresponding scanner or by contacting with the handheld medical device. In addition, it is conceivable that it is detectable via the sensor, which can be a Reed sensor or a metal sensor, such as a Hall probe, whether the coupling of the coupling counterpart to the coupling part is established. Both the sensor and the data memory can be passive components to keep the structural size as small as possible.

One preferred exemplary embodiment of the present invention is explained in more detail hereinafter on the basis of the drawing, in which:

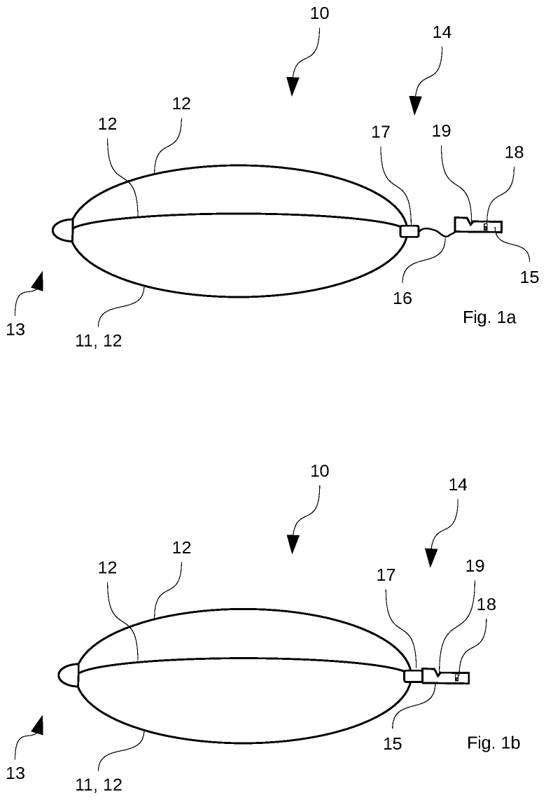

FIG. 1a shows a schematic illustration of an implant,

FIG. 1b shows a schematic illustration of a further exemplary embodiment of an implant,

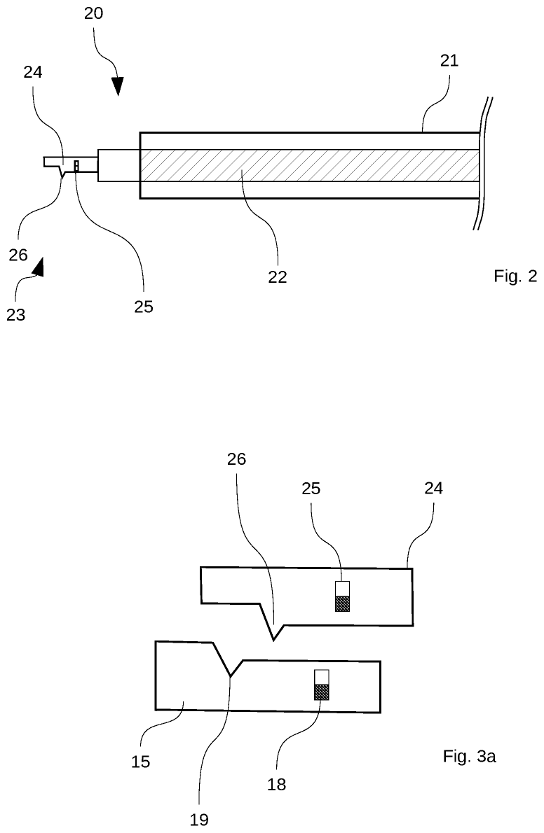

FIG. 2 shows a schematic illustration of a handheld medical device,

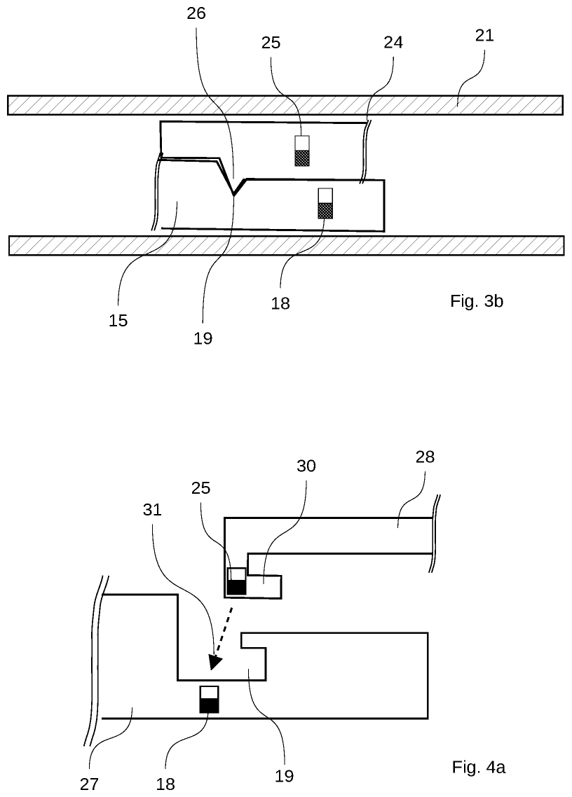

FIG. 3a shows a schematic illustration of an exemplary embodiment of a coupling mechanism,

FIG. 3b shows a further schematic illustration of the coupling mechanism according to FIG. 3a,

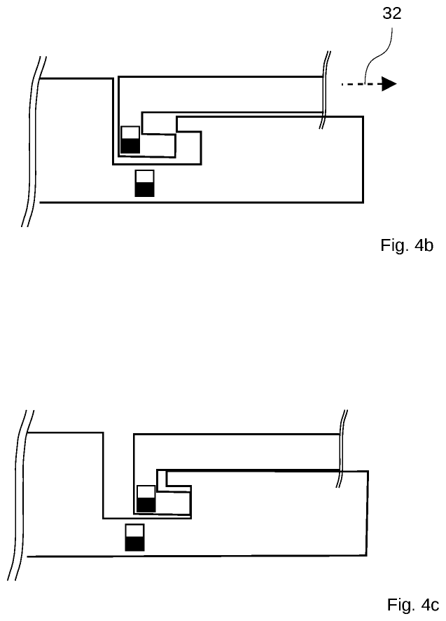

FIG. 4a shows a first schematic illustration of a coupling procedure,

FIG. 4b shows a second schematic illustration of the coupling procedure, and

FIG. 4c shows a third schematic illustration of the coupling procedure.

Possible exemplary embodiments of the invention are schematically shown in the figures. It is to be expressly noted that the invention is not to be restricted to these exemplary embodiments. Rather, it is provided that the invention is also implementable by other embodiments.

An implant 10 is illustrated very schematically in FIG. 1a. This implant 10 consists of a wire structure 11, consisting of three individual wires 12. These wires 12 are connected to one another at a distal end 13 and a proximal end 14 of the implant 10. The wires 12 are produced from a shape memory material, such as Nitinol.

For the treatment, the implant 10 is inserted by a catheter (not shown) in the collapsed state into the urethra of the patient. At the correct position, the wires 12 unfold to form the basket structure shown in FIG. 1a. In addition to the structure illustrated here by way of example, a multiplicity of further shapes is conceivable. Reference is made to the prior art for a further description of the wire structure 11.

For the placement and for the retrieval of the implant 10, it has a coupling part 15 at the proximal end 14. This coupling part 15 is connected via a thread 16 to a proximal end piece 17 of the implant 10. Alternatively to the thread 16, it is also conceivable to use a thin wire. The thread 16 is 1 mm to 10 mm or 5 mm long. A further exemplary embodiment provides that the coupling part 15 is directly connected to the proximal end piece 17 (FIG. 1b).

The coupling part 15 has a ferromagnetic element 18. This ferromagnetic element 18 can be a metal, in particular iron, nickel, or cobalt or a magnetizable alloy or a permanent magnet. A further exemplary embodiment of the invention can provide that the entire coupling part 15 is formed as a ferromagnetic element 18 or as a permanent magnet. In the exemplary embodiment shown in FIG. 1a, the ferromagnetic element 18 is formed as a bar magnet integrated in the coupling part 15. This bar magnet is, for example, embedded during an injection-molding method in a plastic of the coupling part 15. In this molding method, it is also conceivable that the thread 16 is also embedded. Alternatively, it is also conceivable that the thread 16 is connected later to the coupling part 15.

The exemplary embodiment of the coupling part 15 illustrated in FIGS. 1a and 1b additionally has a notch 19. This notch 19 is used as mechanical coupling means. In addition to this notch 19 or alternatively to this notch 19, the coupling part 15 can also have a projection, a slot, a bore, or a pin for a mechanical coupling or contacting.

The implant 10 is coupled to a handheld medical device 20 for the placement and/or retrieval. This handheld medical device 20 is inserted by a catheter 21 into the urethra, as schematically shown in FIG. 2. In this case, the handheld medical device 20 essentially consists of a rod 22, which has a coupling counterpart 24 at a distal end 23. This coupling counterpart 24 is formed complementary to the coupling part 15. The coupling counterpart 24 thus also has a ferromagnetic counter element 25. This ferromagnetic counter element 25 forms a magnetic interaction with the ferromagnetic element 18 of the coupling part 15. For this purpose, it can be formed as a magnetizable metal or as a permanent magnet. If the ferromagnetic element 18 and the ferromagnetic counter element 25 are both formed as permanent magnets, they are oriented in such a way that their polarization is opposing. An attractive magnetic interaction can thus form between the coupling part 15 and the coupling counterpart 24.

A further exemplary embodiment can provide that the ferromagnetic counter element 25 is formed as an electromagnet, which can be switched on and off via the rod 22. In addition, the polarization of the electromagnet may be changed, so that either an attractive or repellent force can be exerted on the ferromagnetic element 18 or the coupling part 15.

The coupling counterpart 24 additionally has a tooth-like projection 26. This tooth-like projection 26 is formed complementary to the notch 19 of the coupling part 15. This projection 26 also represents only one of many possible exemplary embodiments. It is thus also conceivable here that alternatively to the projection 26, the coupling counterpart 24 has a slot, a bore, a notch, a pin, or the like.

In FIG. 3a, the coupling part 15 and the coupling counterpart 24 are shown adjacent to one another without the implant 10 or without the rod 22, respectively, for better illustration. In this position, the ferromagnetic element 18 and the ferromagnetic counter element 25, which are both stylized as permanent magnets, have an attractive effect on one another. This attractive force helps the user of the coupling counterpart 24 to contact with the coupling part 15. As soon as these two parts 15, 24 come into contact with one another, locking of the projection 26 with the notch 19 occurs. This contacting is retained by the permanent magnets. In this coupling state, which is shown very schematically in FIG. 3b, the implant 10 may be both placed in the urethra and retrieved from the urethra by the catheter in a very simple and accurate manner. The coupling part 15 and the coupling counterpart 24 form a circular cross section in the coupled state. Both can thus be pulled together into the catheter 21. The coupling is thus secured against radial displacement in the interior of the catheter 21. It is thus possible to pull with greater force without inadvertently detaching the magnetic coupling.

Due to the minor magnetic interaction between the two permanent magnets, the handheld medical device 20 may be removed from the coupling part 15 after the placement of the implant 10 by minor force action or by a slight rotation of the rod 22. To retrieve the implant 10, it is drawn out through the catheter by means of the handheld medical device 20. At the same time, the wire structure 11 collapses in such a way that it fits into the catheter 21.

To make this procedure particularly gentle to the body, it is additionally provided that a common cross section of the coupling part 15 and of the coupling counterpart 24 is circular or circle-like. An adaptation to the anatomical conditions can thus take place and the placement or retrieval can be carried out without traumatization of the urethra.

A further exemplary embodiment for a coupling part 27 and a coupling counterpart 28 is shown in FIGS. 4a to 4c. For the sake of clarity, the illustration of the implant 10 and of the rod 22 was also omitted here. Precisely as described above, both the coupling part 27 and the coupling counterpart 28 has a ferromagnetic element 18 and a ferromagnetic counter element 25, both being formed as permanent magnets having an opposing polarization. While the coupling part 27 has an indentation 29, the coupling counterpart 28 has a corresponding hook structure 30, which corresponds in a complementary manner to the shape of the indentation 29.

As schematically illustrated in FIG. 4a, the coupling counterpart 28 is attracted by the coupling part 27 in arrow direction 31 due to the magnetic interaction. As soon as the hook structure 30 has been guided into the vicinity or into the indentation 29, the coupling part 27 or the rod 22 is pulled in arrow direction 32 in the proximal direction (FIG. 4b). A mechanical and magnetic connection is formed here between the coupling part 27 and the coupling counterpart 28 (FIG. 4c). In this constellation, the implant may be both placed in a very simple and gentle manner in the urethra and retrieved from the urethra again. The above-described steps are to be carried out in the reverse sequence to decouple the coupling counterpart 28 from the coupling part 27.

### LIST OF REFERENCE NUMERALS

10 implant

11 wire structure

12 wire

13 distal end

14 proximal end

15 coupling part

16 thread

17 end piece

18 element

19 notch

20 handheld medical device

21 catheter

22 rod

23 distal end

24 coupling counterpart

25 counter element

26 projection

27 coupling part

28 coupling counterpart

29 indentation

30 hook structure

31 arrow direction

32 arrow direction

1. An implant for expanding a urethra of a person by means of application of a local ischemic pressure to the tissue of the urethra by a wire structure having at least two wires, the implant being insertable having a distal end in front into the urethra, wherein a proximal end of the implant has a coupling part having a ferromagnetic element, the implant being able to be withdrawn from the urethra via this coupling part.

2. The implant as claimed in claim 1, wherein the ferromagnetic element is a permanent magnet.

3. The implant as claimed in claim 1, wherein the ferromagnetic element is the coupling part.

4. The implant as claimed in claim 1, wherein the ferromagnetic element is integrated in the coupling part part.

5. The implant as claimed in claim 1, wherein the coupling part has at least one projection, one slot, one notch, one bore, one pin, or the like as mechanical coupling means.

6. The implant as claimed in claim 1, wherein the coupling part has a circular cross section or a cross section in the form of a segment of a circle.

7. The implant as claimed in claim 1, wherein a sensor and/or a data memory is arranged in the coupling part.

8. The implant as claimed in claim 1, wherein the coupling part is produced from a plastic.

9. The implant as claimed in claim 1, wherein the coupling part is connected via a wire or a thread or directly to the wire structure.

10. A handheld medical device for positioning and retrieving an implant in a urethra or out of a urethra, respectively, having a rod which is insertable into a catheter the rod having a coupling counterpart at a distal end and the implant being designed as claimed in claim 1, wherein the coupling counterpart has a ferromagnetic counter element for coupling with a coupling part of the implant.

11. The handheld medical device as claimed in claim 10, wherein the ferromagnetic counter element is a permanent magnet.

12. The handheld medical device as claimed in claim 10, wherein the ferromagnetic counter element is the coupling counterpart.

13. The handheld medical device as claimed in claim 10, wherein the ferromagnetic counter element is integrated in the coupling counterpart.

14. The handheld medical device as claimed in claim 10, wherein the coupling counterpart has at least one projection, one slot, one notch, one bore, one pin, or the like as mechanical coupling means, this coupling means being complementary to the coupling means of the coupling part.

15. The handheld medical device as claimed in claim 10, wherein the coupling part of the implant and the coupling counterpart have a common circle-like or circular cross section in the assembled state.

16. The handheld medical device as claimed in claim 10, wherein a sensor and/or a data memory is arranged in the coupling counterpart.

17. The handheld medical device as claimed in claim 10, wherein the coupling counterpart is produced from a plastic.
